(19) United States
(12) Patent Application Publication
(10) Pub. No.: US 2014/0128890 A1
(43) Pub. Date: May 8, 2014

(54) MEDICAL DEVICE AND RELATED METHODS OF USE

(71) Applicant: Boston Scientific Scimed, Inc., Maple Grove, MN (US)

(72) Inventors: Gary KAPPEL, Acton, MA (US); Norman MAY, Northborough, MA (US); Douglas MELANSON, Natick, MA (US)

(73) Assignee: Boston Scientific Scimed, Inc., Maple Grove, MN (US)

(21) Appl. No.: 14/047,245

(22) Filed: Oct. 7, 2013

Related U.S. Application Data
(60) Provisional application No. 61/721,874, filed on Nov. 2, 2012.

(51) Int. Cl. A61B 17/062; A61B 17/04

(52) U.S. Cl. CPC A61B 17/0625; A61B 17/0469
USPC 606/147

(57) ABSTRACT

A medical needle driver including an elongated member having a proximal end, a distal end, and a lumen extending therebetween. The medical needle driver may also include an end-effector disposed at the distal end of the elongated member, the end-effector may include at least two arms connected at their proximal ends to form a jaw-like structure, wherein at least one of the at least two arms may be configured to rotate relative to the other arm. In addition, each arm may include an inner face, the inner face of at least one of the arms including at least one groove configured to receive a portion of a needle, wherein the groove may include a first geometric structure configured to mate with a corresponding second geometric structure on the needle.

### CROSS-REFERENCE TO RELATED APPLICATIONS

This application claims the benefit of priority from U.S. Provisional Application No. 61/721,874, filed on Nov. 2, 2012, the entirety of which is incorporated by reference herein.

### FIELD

Embodiments of the present disclosure relate generally to medical devices and procedures. In particular, embodiments of the present disclosure relate to minimally invasive medical devices for manipulating, e.g., medical tools inside a patient's body.

### BACKGROUND

Minimally invasive medical procedures, such as endoscopy and the like, are carried out by inserting a surgical tool into the patient's body through small incisions or anatomical openings (e.g., the oral, vaginal, and/or anal cavities) to perform surgery or conduct a diagnostic or other procedure, while causing minimal tissue trauma and avoiding the large incisions typically needed for “open” surgeries. The current state of this art and its future directions are pushing designers to produce devices and tools that are ever smaller, without sacrificing actuation and gripping forces when compared to presently known devices. These devices, however, must still be able to complete their expected tasks, such as gripping, controlling, and/or driving a needle or other tools.

Certain minimally invasive surgical procedures may require suturing, which must be accomplished at the surgical site, using needle drivers, which can be end-effectors carried on endoscopic devices. Typically, surgeons employ a number of needle configurations for suturing during a minimally invasive surgical procedure. In some instances, the needles may be curved or straight. A third variety, called a ski-needle, combines both types, as the ski-needle is curved at the proximal (suturing) end but straight at the distal end, which can be used to manipulate or drive the needle. Curved needles are sometimes used in endoscopic or other minimally invasive procedures involving delicate or fine tissue or space restrictions, as they require a relatively minimum amount of movement and space. A needle driver, also referred to as a needle holder, is a device used to hold or control surgical needles while suturing during minimally invasive procedures.

Various types of needle drivers are available, allowing an operator to manipulate both straight and curved needles. Conventional needle drivers, however, require the operator to maintain pressure on the needle to grip the needle, and occasionally, proper grip and pressure are not achieved. Thus, needle movement may cause inconvenience to the surgeon and may also damage the surrounding tissues.

During surgical procedures, the need remains for precision needle drivers, which are able to grasp a needle firmly with an end-effector to ensure safe, fast, and successful outcomes. Additionally, the time and effort required to use needle drivers and needles during surgery may also be a factor during a minimally invasive surgical procedure. Thus, there is a need for improved needle driver end-effectors to optimize the grip, control, and drive of the needle during minimally invasive surgical processes.

### SUMMARY

Embodiments of this disclosure relate generally to medical devices and procedures. In particular, embodiments of the present disclosure relate to surgical needle drivers having end-effectors that can be used to grasp, drive, and control surgical needles.

In one embodiment, a needle driver may include an elongated member having a proximal end, a distal end, and a lumen extending therebetween. The medical needle driver may also include an end-effector disposed at the distal end of the elongated member, the end-effector may include at least two arms connected at their proximal ends to form a jaw-like structure, wherein at least one of the at least two arms may be configured to rotate relative to the other arm. In addition, each arm may include an inner face, the inner face of at least one of the arms including at least one groove configured to receive a portion of a needle, wherein the groove may include a first geometric structure configured to mate with a corresponding second geometric structure on the needle.

Various embodiments of the medical needle driver may include one or more of the following features: the at least one groove may include a plurality of grooves; the inner face of at least one of the arms may include a substantially flat surface disposed adjacent the at least one groove; the needle may be an arcuate needle and each of the plurality of grooves may be curved to correspond to the arcuate needle; the first geometric structure may be a projection; the second geometric structure may be a cavity configured to receive the first geometric structure; the groove may include a plurality of first geometric structures; the needle may include a plurality of second geometric structures; and both arms of the end-effector may be configured to rotate relative to one another; the needle may be configured to be secured to a length of suture.

In another embodiment, a needle driver may include an end-effector disposed at the distal end of the elongated member; the end-effector may include at least two arms pivotally connected at their proximal ends. In addition, each arm may include an inner face, the inner face of at least one of the arms having a plurality of grooves configured to receive a portion of a needle and a substantially flat surface disposed adjacent the plurality of grooves, and wherein each of the plurality of grooves may include a projection configured to mate with a corresponding recess on the needle.

Various embodiments of the medical needle driver may include one or more of the following features: the needle may be an arcuate needle and each of the plurality of grooves may be curved to correspond to the arcuate needle; the projection may be a plurality of projections; both arms of the end-effector may be configured to rotate relative to one another; the end-effector may be secured to a distal end of an endoscopic tool; the needle may be configured to be securely attached to a length of suture; and the substantially flat surface may include surface features to increase friction during gripping.

In a further embodiment, a needle driver may include an elongated member having a proximal end, a distal end, and a lumen extending therebetween. The needle driver may also include an end-effector disposed at the distal end of the elongated member; the end-effector may include two arms connected at their proximal ends to form a jaw-like structure, wherein each of the two arms is configured to rotate relative to the other arm. Further, each arm may include an inner face, the inner face of both arms including a plurality of groove configured to receive a portion of a needle, wherein each of the plurality of grooves may include a first geometric structure configured to mate with a corresponding second geometric structure on the needle.

Various embodiments of the medical needle driver may include one or more of the following features: each of the plurality of grooves may include a plurality of geometric structures; and each of the inner faces may include a substantially flat surface disposed adjacent to the plurality of grooves. In some embodiments, the needle may be shaped or may include features that are complimentary to and/or correspond to the groove and/or geometric structures of the needle driver.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate embodiments of the disclosure and together with the description, serve to explain the principles of the disclosure.

FIGS. 1A-1B are perspective views of a needle driver end-effector in the open and the closed positions, in accordance with an embodiment of the present disclosure.

FIGS. 2A-2B are side views of the exemplary needle driver end-effector of FIGS. 1A-1B in the open and closed positions, respectively.

FIGS. 3A-3B are side views of another exemplary needle driver end-effector in open and closed positions, in accordance with a further embodiment of the present disclosure.

FIG. 4 depicts a curved needle configured for performing minimally invasive medical procedures.

FIGS. 5A-5D depict exemplary needles, in accordance with other embodiments of the present disclosure.

FIG. 5E depicts an exemplary needle having a flat section to aid gripping.

FIG. 6 is a top view of a lower arm of a needle driver end-effector holding a needle, in accordance with an embodiment of the present disclosure.

FIG. 7A is a top view of the lower arm of a needle driver end-effector holding a needle, in accordance with another embodiment of the present disclosure.

FIG. 7B is a top view of an inner face of a lower arm of a needle driver end-effector, in accordance with yet another embodiment of the present disclosure.

FIG. 7C is a cross-sectional view of the needle driver of FIG. 7B holding the needle.

FIG. 8 illustrates a portion of an exemplary face of one of the arms of a needle driver end-effector, in accordance with some embodiments of the present disclosure.

FIGS. 9A-9B illustrate a needle driver end-effector with exemplary surface features, in accordance with some embodiments of the present disclosure.

FIGS. 10A-10B illustrate another exemplary needle driver end-effector, in accordance with another embodiment of the present disclosure.

### DETAILED DESCRIPTION

Reference will now be made in detail to embodiments of the disclosure, examples of which are illustrated in the accompanying drawings. Wherever possible, the same reference numbers will be used throughout the drawings to refer to the same or like parts.

The present disclosure provides an improved needle driver having an end-effector with one or more features to optimize gripping of the needle, to control and drive the needle. A medical device including the needle driver having an end-effector and a number of associated needles are disclosed. The needle driver may include an elongated member having a proximal end, a distal end, and a lumen extending therebetween. The elongated member may further include an end-effector disposed at its distal end. The end-effector may form a jaw-like structure having two arms rotatably connected to move between open and closed configurations, actuated by a control member connected to a proximal portion of one or both arms and extending through the elongated member to a controller (not shown). Various configurations of end-effector actuation, structure, and function are described in the embodiments of the disclosure. Further, as used in this disclosure, “distal” refers to a position or direction further from the user, and “proximal” refers to a position or direction opposite “distal” and closer to the user.

Each of the end-effector arms may include an inner face and an external face, the inner face of the arms may include at least one ridge or at least one V-shaped slot or groove for holding a needle. The slot may have any suitable configuration. The needle may include at least one notch corresponding to the ridge formed on the end-effector of the needle driver or a flat section. The two arms may be differentiated as an “upper” and “lower” arm or jaw.

More particularly, the present disclosure provides a medical device including a needle driver having an end-effector for gripping and controlling a needle during minimally invasive procedures. In addition, because the end-effector is not limited to grasping a suturing needle, the end-effector of the present disclosure is also useful for securely holding and manipulating tissues or other tools as appropriate.

### EXEMPLARY EMBODIMENTS

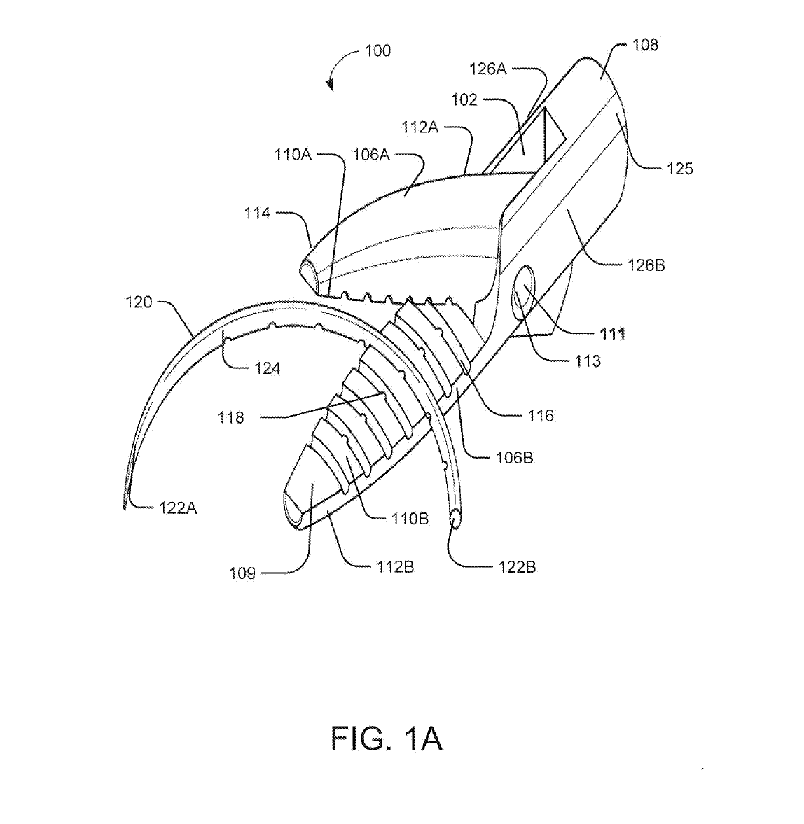

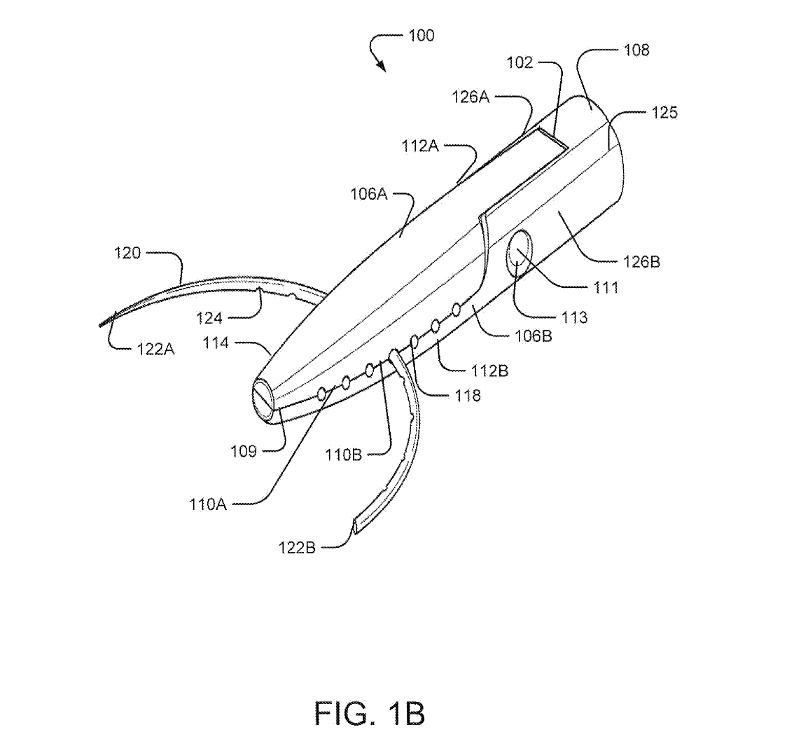

FIG. 1A is a perspective view of an exemplary needle driver end-effector 100 (also referred to as “end-effector 100”) in an open configuration, in accordance with an embodiment of the present disclosure. The needle driver end-effector 100 is a medical device that may be used to hold, control, or drive a surgical needle 120 during a medical procedure within a patient's body. The end-effector 100 may include, among other things, a clevis 108, which may be configured to be secured to a distal portion of an elongated member (not shown). The clevis 108 may include a base portion 125 and two leg portions 126A-126B separated by a slot 102 that has at least one pivot pin hole 111 at each end of the leg portions 126A-126B for accepting at least one pivot pin 113. In addition, the end-effector 100 may include at least two jaws or arms 106—an upper arm 106A and a lower arm 106B, (hereafter, arms 106)—pivotally connected within the slot 102 of the clevis 108 by the pivot pin 113. In another embodiment, as depicted in FIG. 1A, one of the arms 106, such as, e.g., arm 106B may integrally extend from the clevis 108. A control member (not shown) extends through the elongated member, translating the movements from a controller or handle (not shown) present at the proximal end of the elongated member. The control member (not shown) may be operably connected to the proximal end of the upper arm 106A, so that actuating the upper arm 106A at its proximal end translates into movement of the upper arm 106A at their distal end 114. The control member may be also operably coupled to proximal ends of both arms 106A-106B.

The upper arm 106A may include an inner face 110A and the lower arm 106B may include an inner face 110B. Here, both inner faces 110A and 110B will be collectively referred as inner faces 110. As shown in FIG. 1A, the inner faces 110 of both arms 106 may include a substantially flat surface 109. The flat surface 109 may be disposed at a distal portion of inner faces 110, a middle portion, or a proximal portion. In an embodiment of the present disclosure, any or all of the inner faces 110 may be substantially flat like the flat surface 109. Each of the arms 106 also has an outer face 112A-112B (hereafter, outer faces 112) respectively. The inner faces 110 of the arms 106 may include a number of channels or grooves 116 of suitable construction and dimension. The cross-section of the grooves 116 may be curved or radial. Alternatively, in other embodiments, the cross-section of grooves 116 may be rectangular, semicircular, triangular, V-shaped, or a combination of shapes that prove to be effective in the intended environment for the desired function.

Each of the grooves 116 may further include a number of projections, such as, e.g., ridges 118 configured to receive and secure the needle 120 or suture relative to arms 106. The ridges 118 may be keying or indexing ridges. In another embodiment of the present disclosure, the ridges 118 may be serration grooves perpendicular to the needle, which may allow the arms 106 to grip the needle 120 or suture more firmly. The ridges 118 may be configured to secure a needle in place relative to the respective arms 106, and to prevent the needle 120 from slipping or changing its position while performing minimally invasive procedures. The ridges 118 may be present on inner face 110 of one or both of arms 106. The shape, dimension, size, and number of ridges 118 may vary depending on the structure or shape of the needle 120. Alternatively, in other embodiments, the cross section of the ridges 118 may be rectangular, semicircular, a combination of shapes or any shape or combination of shapes that prove to be effective in the intended environment for the desired function. The ridges 118 may also lie parallel to one another on any axis in a lateral plane. Additionally, the ridges 118 may also intersect. Alternatively, the ridges 118 may be present on both arms 106, seated within grooves 116. Ridges 118 of various shapes, sizes, and orientations may coexist on the same inner face 110. For example, some of the ridges 118 may be larger than others. Alternatively, the ridges 118 may have a sharp or blunt face based on the texture and properties of the needle to be manipulated. Further, the ridges 118 may be disposed opposing or alternating one another within the grooves 116. Although, FIG. 1A shows only one ridge 118 on each of the grooves 116, a person skilled in the art will appreciate that each of the grooves 116 may include more than one ridge 118. Moreover, the cross section of the ridges 118 may have an angular slope, such as the “V”-shaped ridges, as shown in FIG. 8.

At their distal end 114, the arms 106 may be linear or curved. FIG. 1A shows only one arm, the upper arm 106A, which is capable of pivoting. It will be understood to a person skilled in the art that both arms 106 may pivot or rotate. In some embodiments, arms 106 may pivot and rotate relative to each other or relative to the clevis 108. Further, the arms 106 may be configured to grip tissue as well.

Further, the arms 106 may include a locking mechanism to fix them in a specific position relative to each other. Here, a locking mechanism may include any suitable mechanism, such as snap fit, screw, or fastening mechanism. The arms 106 may also include a magnetic element (not shown), which may lock the arms by the resulting magnetic forces. In some embodiments, the needle 120 may also have keying or interlocking features across the upper arm 106A and/or the lower arm 106B.

In alternate embodiments of the present disclosure, the inner faces 110 of the arms 106 may be removably coupled to each of the arms 106. The arms 106 may include locking mechanisms to which the inner faces 110 may be attached. Alternatively, the arms 106 may also be detachable from the clevis 108. In additional embodiments of the present disclosure, the distal end 114 of the upper arm 106A (or top arm) may be varied in shapes and sizes. For example, a spear-headed tip may be employed, which may be used in skewering obstructing or unwanted tissue. The sharp end may also be used to tear open tissue to reach otherwise inaccessible areas in the body. The distal end 114 may also be shaped as a curved beak, or it may be tapered laterally, giving rise to a flattened edge. The flat surface 109 may also be beveled to prevent injury to the tissue. The modified distal ends may also be used for scraping plaque or debris adhered to the tissue. The modified distal ends may also be used as a wedge to separate tissue layers or to single out vascular structures from a bundle. The distal end modifications may be present either on the lower arm 106B or on both the arms 106. The inner face 110 on at least one of the arms 106 may also include bisection along the length of the arms 106. The bisection may take the form of a channel, allowing an element such as a blade to move forward to the distal end 114.

The needle driver end-effector 100 may also include an advancing member, such as, e.g., a cutter tool. In addition, the cutter tool may be any other tool, such as an electro-cautery blade, a coagulation forceps, suction cautery devices, laparoscopic electrodes, laser fibers, lithotripters, and electrode cautery tips, which may include ball-tip, needle, and extended or flat blade electrodes.

The cutter tool may be a cautery blade, which is present at the proximal side of the distal end 114 of the arms 106. The cutter tool may be connected to the control member (not shown), and may be actuated by articulating the controller (not shown) present at the proximal end of the control member. If connecting cutter tool to the existing controller proves inconvenient, then an additional control member (not shown) may be provided.

Further, the inner faces 110 of each of the arms 106 may also include micro features having, e.g., back angled cuts on a left half facing left of the arms 106 and on a right side facing the right of the arms 106 to pull the needle in either direction. Alternatively, those of skill in the art will see that the surface could be optimized for movement in all directions. The end-effector 100 may also have geometric features that can mate with corresponding geometric features on the needle to decrease chances of the needle slipping during use in minimally invasive surgery. Further, the flat surface 109 towards the distal end 114 may have an unridged portion having a frictional face. The inner faces 110A-110B on both the arms 106 may have the flat surface 109A-109B, respectively.

FIG. 1B is a perspective view of the end-effector 100 of FIG. 1A, depicted in a closed position. The end-effector 100 may be opened by actuating the controller or control member and closed for gripping or holding the needle 120. The outer faces 112A-112B of the distal end 114 may be of any suitable shape that provides an atraumatic face, such as, e.g., beveled edges and rounded corners. Further, the distal end 114 may include a pointed tip. The inner faces 110 may be formed of insulated or non-conductive material, such as ceramic, plastic or any other suitable material known in the art. Further, in an embodiment, the inner faces 110A-110B may have a high durometer elastomer or plastic to aid the gripping of the needle 120 or a suture. In other embodiments of the present disclosure, the outer faces 112 may be made of any rigid, biocompatible material, which is also atraumatic to tissue, such as metals, plastics, ceramics, and so forth. The movement of the control member or actuator may apply force to the proximal end of the arms 106, moving the arms 106 radially outward from a collapsed or closed state to an expanded or an open state. In some embodiments, the needle driver end-effector 100 may have a thinner upper arm 106A. Thus, reduced surface area of the upper arm 106A may allow for a higher holding force. Further, the arms 106A-106B may have carbide or other hardened or sharpened insets to allow for better gripping of the needle 120.

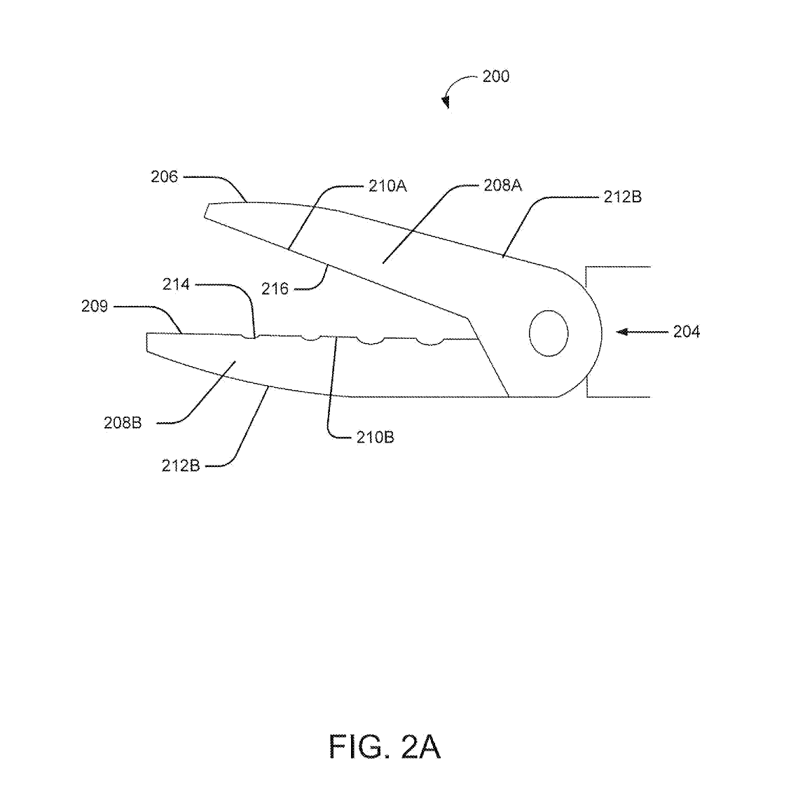

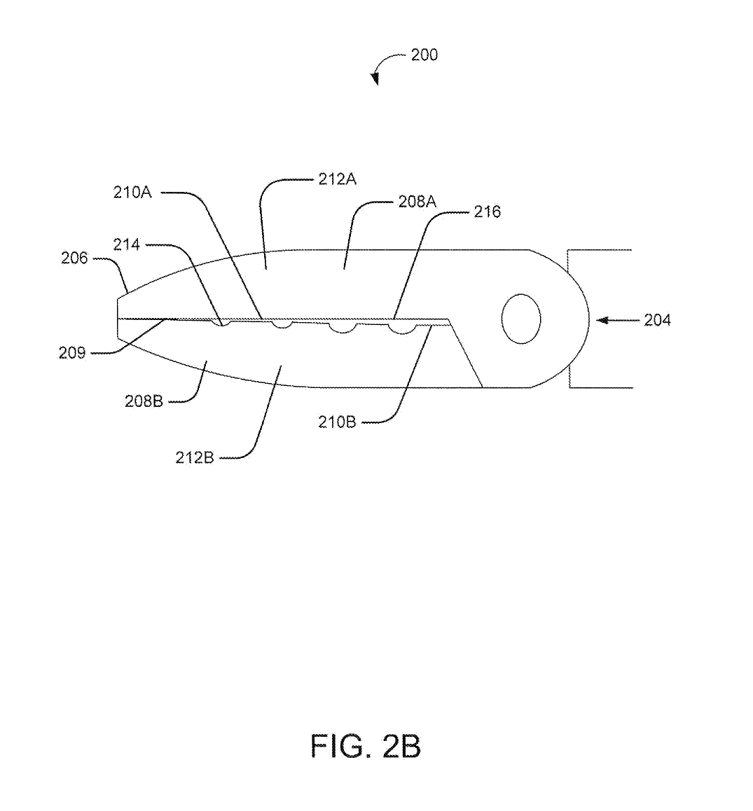

The end-effector 100 may grip or control different types of needles such as the needle 120. The needle 120 may include a distal end 122A and a proximal end 122B. The distal end 122A may be a pointed tip to pierce tissue while performing various medical procedures. Further, the proximal end 122B is configurable to be secured to a suture thread. In addition, the proximal end 122B may have an opening or eye for receiving the suture thread. The needle 120 may have one or more notches or splits 124 on at least one of its inner face or outer face that correspond to the ridges 118 of the end-effector 100. The notches 124 may interlock with the ridges 118 within the end-effector 100. The upper arm 106A may have curved grooves or a flat surface to apply pressure on the needle 120, as described in detail in connection with FIGS. 2A-2B.

FIG. 2A is a side view of an exemplary needle driver end-effector 200, depicted in an open configuration, in accordance with an embodiment of the disclosure. The end-effector 200 is similar to the end-effector 100, as shown in FIGS. 1A-1B, with a structure similar to that of the needle driver end-effector 100. Arms 208A and 208B (hereinafter, referred collectively as arms 208) of the end-effector 200 have inner faces 210A-210B and outer faces 212A-212B, respectively. The inner face 210A of the upper arm 208A may be a flattened face 216 to apply pressure on a needle, such as the needle 120 shown in FIGS. 1A-1B. The inner face 210B of the lower arm 208B may include a number of grooves 214 for receiving a needle. In some embodiments, the grooves 214 may be curved. For example, the grooves 214 may be radial grooves including one or more ridges or pockets to lock-in the needles in the end-effector 100. Further, the inner face 210B may include an unridged portion having a frictional face 209. The end-effector 200 may be secured to a distal portion of an elongated member having a proximal end 204 and a distal end 206.

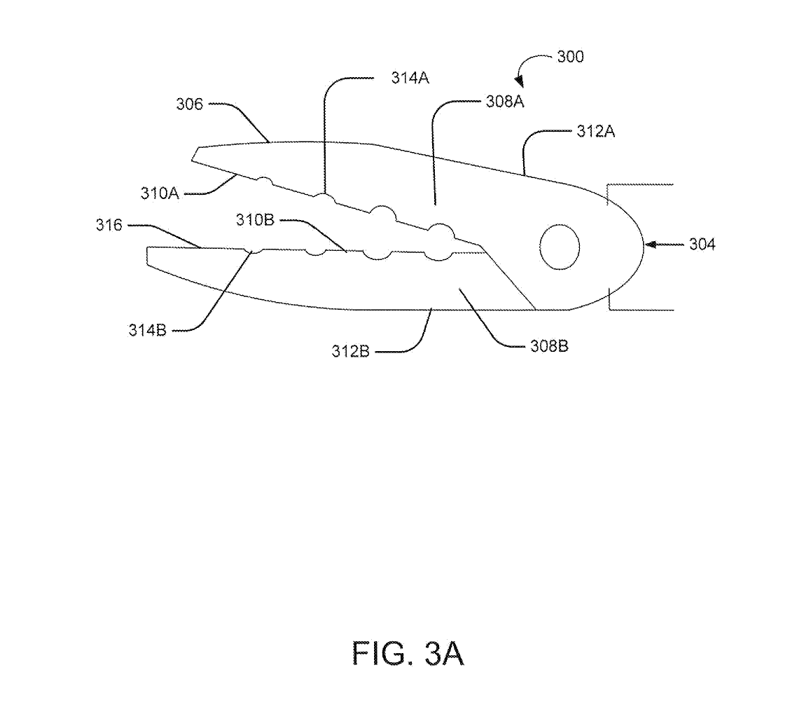

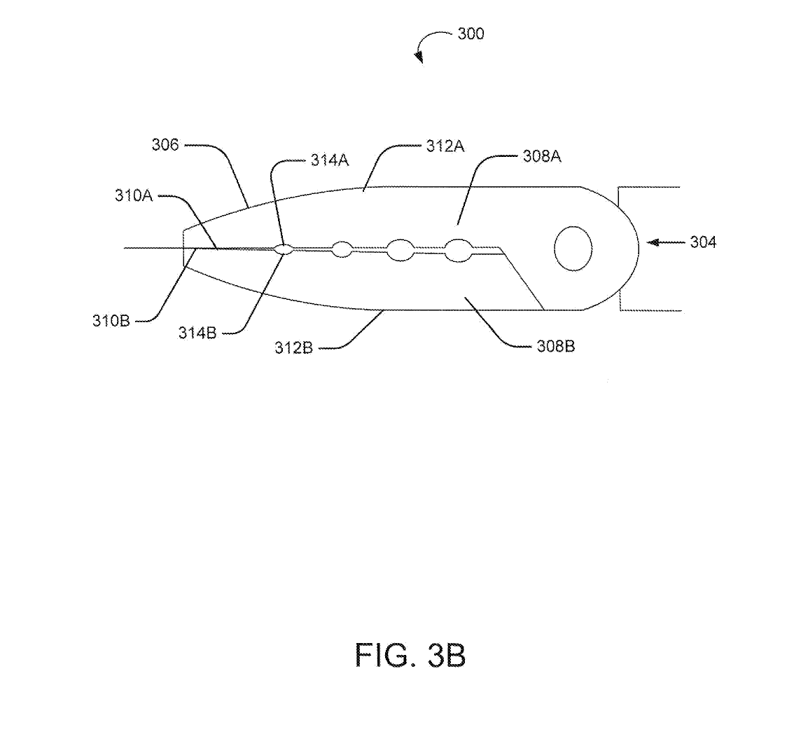

FIG. 2B illustrates the exemplary needle driver end-effector 200, depicted in a closed configuration. A control member, or multiple control members, may be operably coupled to the proximal end of the arms 106 for actuating the movement of the arms 208. Further, both the arms 208 may include grooves 214 of any suitable configuration as described in detail in connection with FIGS. 3A-3B. FIG. 2B also shows a variable gap between the upper and lower arms 208A, 208B and variable size grooves 214 along the inner faces 210A, 210B to accommodate various diameter needles. The size and variance of the gap and the grooves are just examples, the relative alignment. The arrangement is not so limited and it is contemplated that the gap and grooves may have any size and/or variance.

FIG. 3A is a side view of another exemplary needle driver end-effector 300, depicted in an open configuration or state, in accordance with another embodiment of the present disclosure. The end-effector 300 is similar to the end-effector 100, as shown in FIGS. 1A-1B, with a structure similar to that of the needle driver end-effector 100. The needle driver end-effector 300 includes at least two arms 308—an upper arm 308A and a lower arm 308B. Each of the arms 308A and 308B may include inner faces 310A-310B and outer faces 312A-312B, respectively. The inner faces 310A-310B of the arms 308A-308B may include one or more grooves 314A-314B, respectively. The grooves 314 can be curved grooves or radial grooves or may be of any suitable geometric shape for holding one or more needles in place. The inner faces 310A-310B may also include an adhesive coating, depressions, or slots to accommodate a needle. The inner faces 310A-310B may also be made of material such as fabric, plastic, rubber, ceramic, or metal. The present embodiment may further be used along with the previous embodiments.

Each of the grooves 314 may include ridges (not shown) that may be present at a proximal or distal region, or alternatively may be present on both the proximal and distal regions of the arms 308, and there may be a patch or region along the inner faces 310A-310B that is free from any ridges (“unridged portion 316”). The unridged portion 316 may include a non-slippery face or frictional face, which in turn may have a roughened face to increase friction. FIG. 3B depicts the side view of the end-effector 300 in the closed configuration or state.

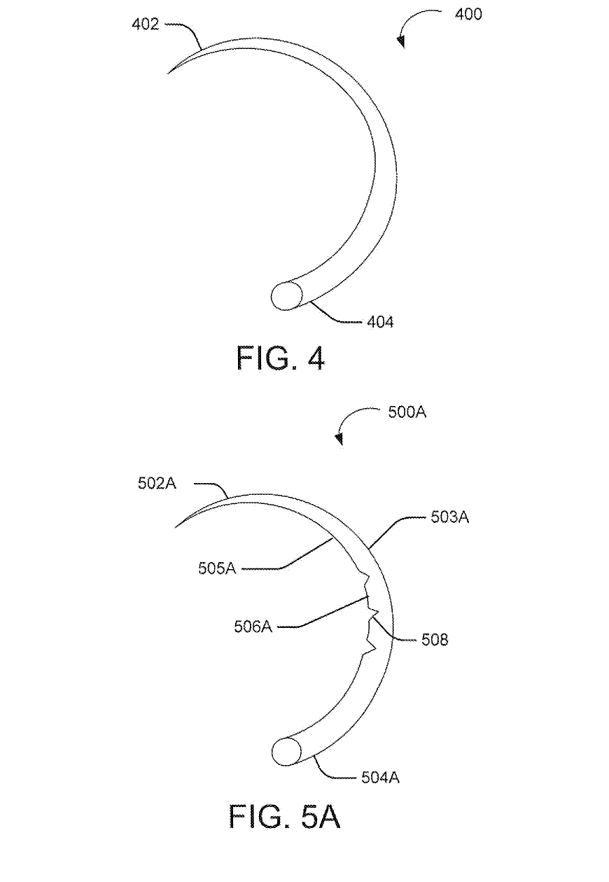

FIG. 4 depicts a curved needle 400 used in minimally invasive surgical procedures for suturing surgical incisions or more generally for closing wounds or draining tissue. The needle 400 is shown curved, which shape is widely used for surgical suturing, because that shape allows the surgeon to insert the needle on one side of the incision and easily pass the needle under the incision and through tissue on the other side, as can be readily envisioned. However, any suitable shape or configuration may be used. A distal end 402 of the needle is adapted to penetrate tissue, and to that end, it may be tapered to a point, or formed to a sharpened cutting edge. For suturing friable tissue, a blunt needle may prove most useful. A proximal end 404 of the needle 400 is adapted to receive the suturing thread, and surgical needles are provided in a reusable or one-time use form. Reusable needles include an eye at their proximal end, through which the surgeon feeds the suture thread. A single-use needle is generally provided with the suturing threads swaged or otherwise attached. Surgical needles are provided in a wide variety of length, curvature, and specific structure, tailored to particular surgical scenarios. Those in the art will understand that the disclosure below builds upon general needle structure to achieve the innovations described herein.

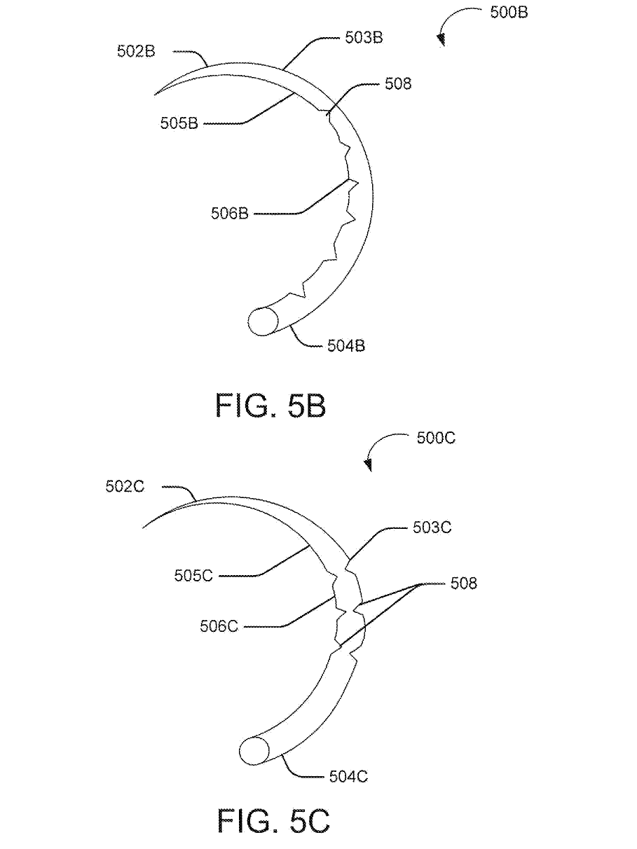

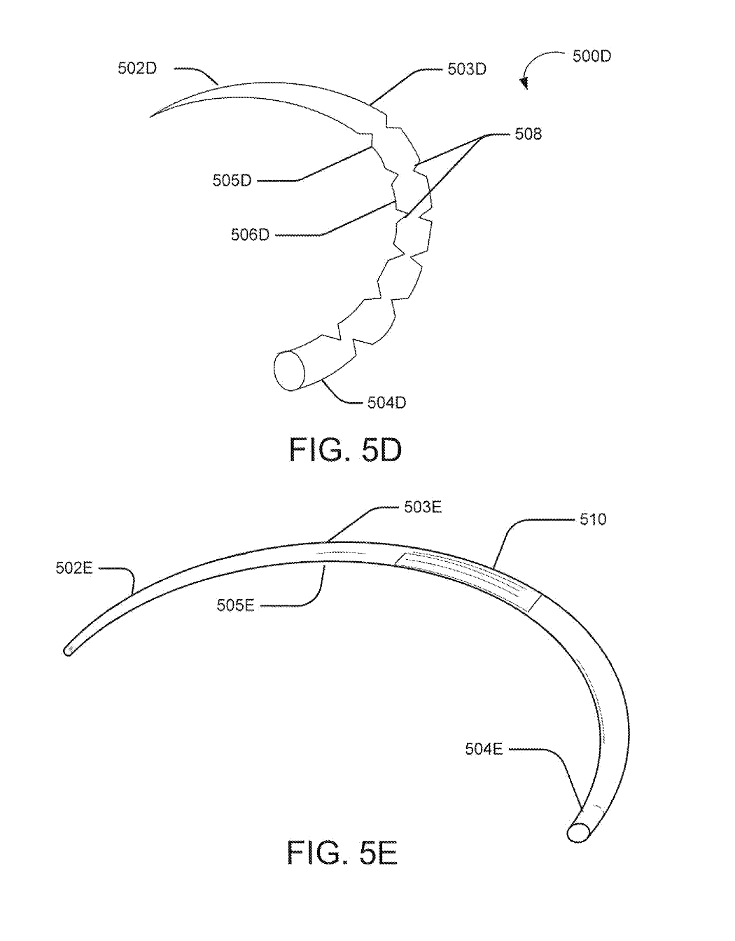

FIGS. 5A-5D depict exemplary needles having notches, in accordance with various embodiments of the present disclosure. FIG. 5A depicts a needle 500A having a distal end 502A and a proximal end 504A. The needle 500A may be curved in shape and may have a circular cross-section. A person skilled in the art will appreciate that the shape and cross-section of the needle 500A may differ depending on the type of needle used in different surgical procedures. For example, the cross section of the needle 500A may be triangular, rectangular, and so forth. The distal end 502A of the needle 500A may be a sharp and pointed tip that may be used to perform various medical procedures such as suturing. The needle 500A has an outer surface 503A and an inner surface 505A. The proximal end 504A may include an eye for including or securing the suture thread. The needle 500A also includes a gripping section 506A, adapted to provide an enhanced gripping surface, for holding the needle securely in a needle driver during certain procedures. In addition, the needle 500A may have keying or interlocking features across the inside face 505A. In some embodiments, the needle 120 may include a knurled section (not shown) or other features to aid gripping.

Conventional needle drivers typically are rigid, solid shaft devices that can apply much greater actuation or clamping forces to the end-effector jaws or arms as compared to the flexible shaft devices. The disclosed end-effector 100 with the keying and interlocking features on the arms 106 may allow lower actuation forces to be applied while maintaining functionality of the conventional needle drivers. In some embodiments, the needle driver end-effector 100 may have a thinner upper arm 106A, which may allow for a higher force holding force.

As shown, the needle 500A includes a number of notches 508 on its inner face 505A. The notches 508 may be angular or “V”-shaped cuts or slits on surface of the needle 500A. The gripping section 506A may occupy only a portion, such as, e.g., a central region, proximal region, a distal region, or any combination thereof, of needle 500A, as desired.

FIG. 5B depicts a needle 500B likewise including a gripping section 506B. Here, gripping section 506B extends completely over an inner face 505B of the needle 500B, having keying or interlocking features in form of notches 508 or similar structures, as noted above, spaced along its length. The needle also has a smooth surface and pointed tip towards a distal end 502B. An outer face 503B is also smooth and may not include any notches 508.

In some embodiments, the needle 500A may have keying or interlocking features across both faces. FIG. 5C depicts a needle 500C having keying or interlocking features in form of notches 508 on both faces i.e., an inner face 505C and an outer face 503C. The needle 500C may have a pointed distal end 502C and a proximal end 504C. The needle 500C may also have a gripping section 506C including the notches 508.

A further alternative is shown in FIG. 5D, which depicts a gripping section 506D extending completely over the entire surface. Here, the notches 508 are cut into both the faces i.e., an inner face 505D and an outer face 503D, forming the gripping section 506D. In some embodiments, the needle 500D may have keying or interlocking features across the inner face 505D and the outer face 503D. The notches 508 of the needles 500A, 500B, 500C, and 500D may lock into the male ridges 118 of the needle driver end-effector 100, as described in detail in connection with FIGS. 1A-1B.

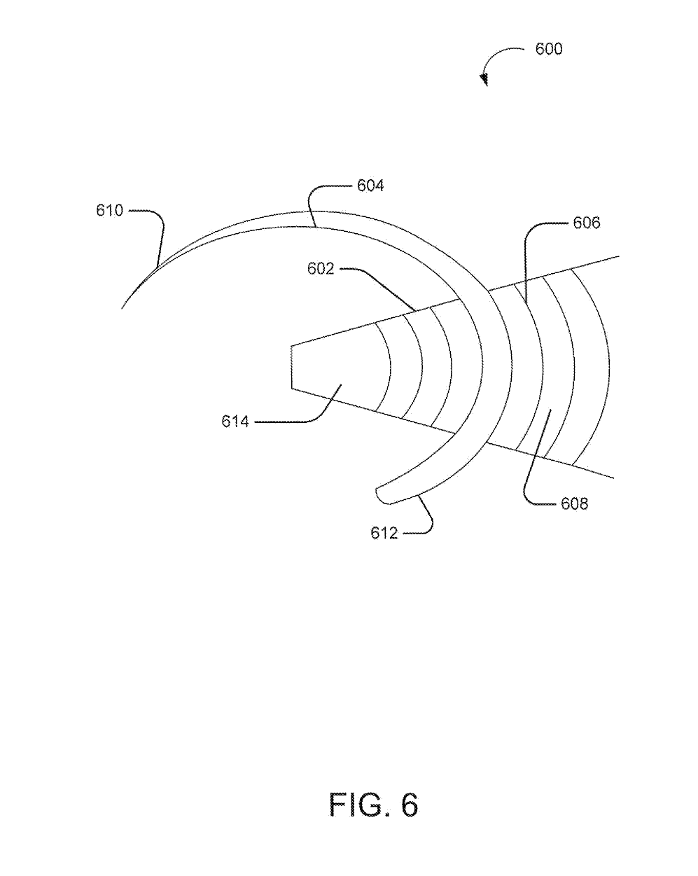

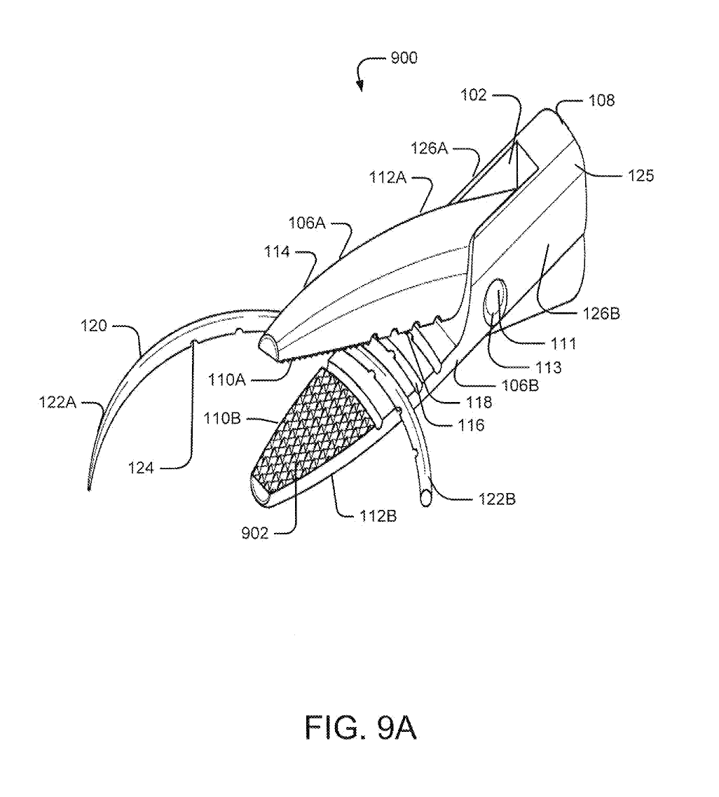

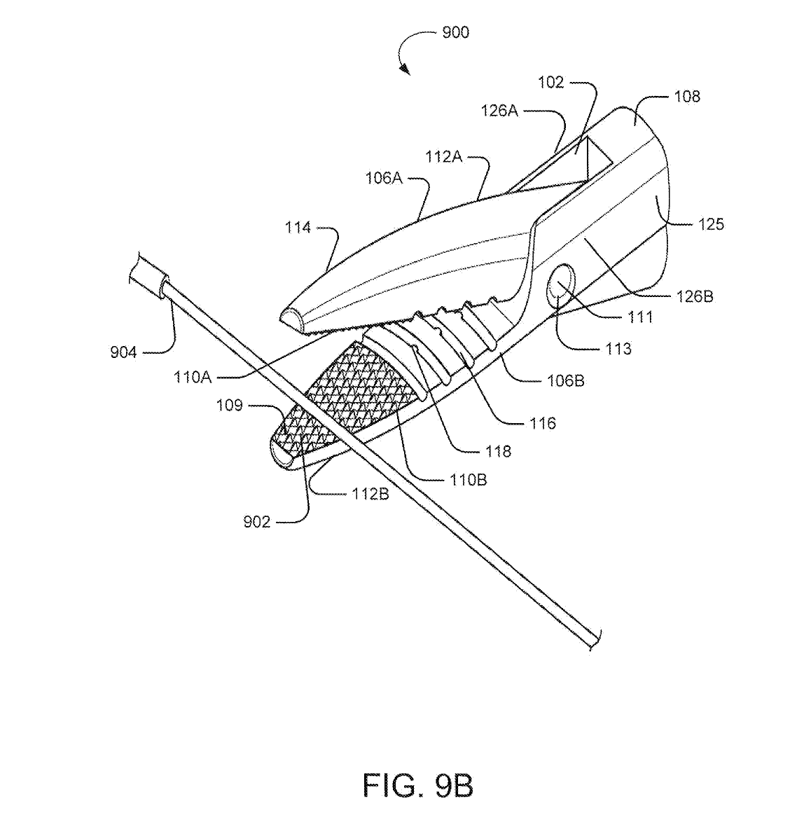

FIG. 5E illustrates an exemplary needle 500E including a flat section 510 to aid gripping. The needle 500E has a distal end 502E and a proximal end 504E. The needle further includes an inner face 505E and an outer face 503E. The flat section 510 may be present in the middle of the needle 500E. In some embodiments, the flat section 510 may be present towards the proximal end 504E. FIG. 6 is a top view of a lower arm 602 of a needle driver end-effector 600 holding a needle 604 having a distal end 610 and a proximal end 612. In addition, here the needle surface extending between the distal end 610 and the proximal end 612 may be substantially smooth. The needle driver end-effector 600 may include two arms, similar in form and function to a pair of pliers or arms as shown in FIGS. 1A-1B. An inner face 608 of the lower arm 602 may include a number of grooves 606, sized and formed to accept the needle 604. In the illustrated embodiment, for example, the needle 604 is curved to a particular radius, and groove 606 is formed to match that shape. Though not shown, the upper arm may also include similar grooves, allowing the surgeon to hold a needle 604 while suturing. Further, the inner face 608 may have a flat surface 614 disposed toward the distal end of the arm 602. Formation and employment of such surfaces is discussed below in connection with FIGS. 9A and 9B. In any of the embodiments described herein, the curve of the groove may substantially approximate the curve of the needle; however, it may not be an exact approximation. As such, the difference in shape and the resulting unalignment may assist in retaining the needle in a corresponding groove by reducing the likelihood that the needle will slide within the groove.

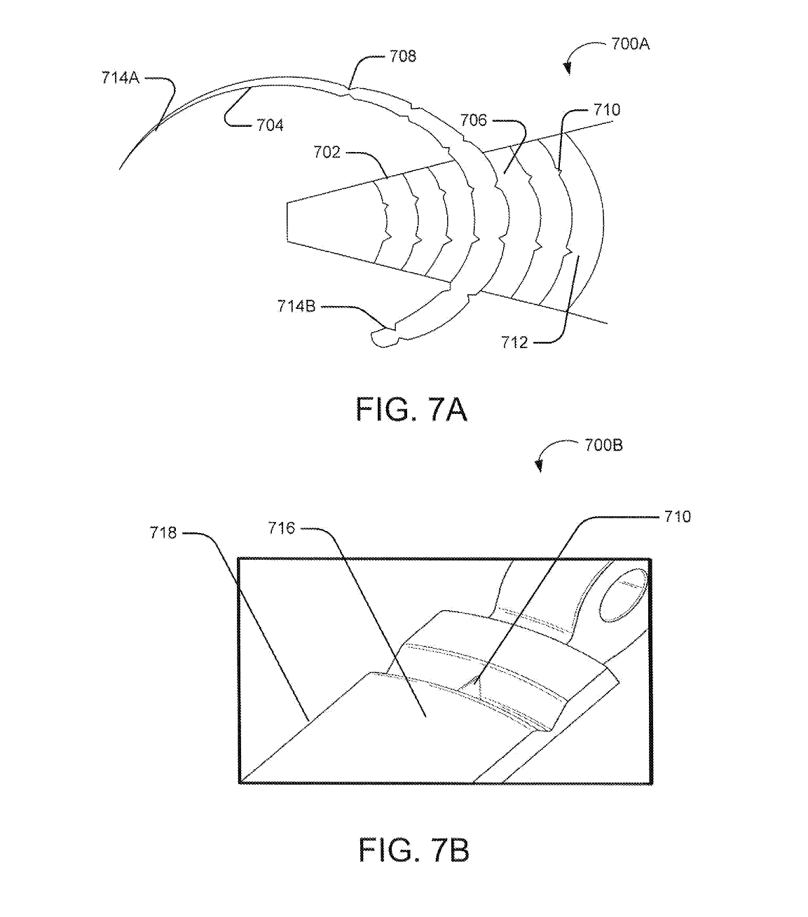

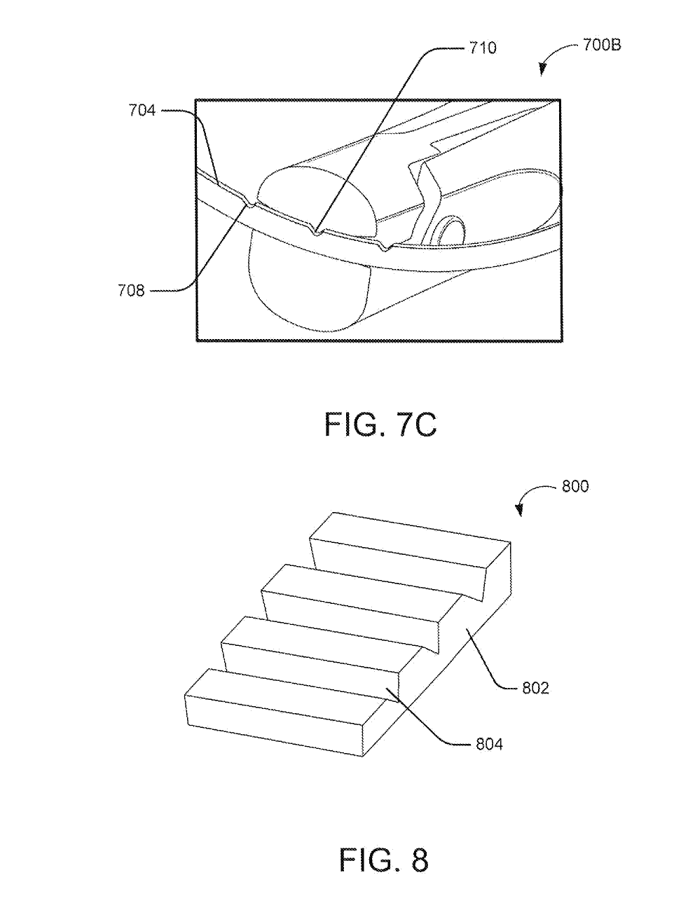

FIG. 7A is a top view of a lower arm 702 of a needle driver end-effector 700A holding a needle 704, in accordance with another embodiment of the present disclosure. Like several embodiments discussed above, this implementation includes one or more grooves 706 in lower arm 702, but here the grooves 706 include ridges (or posts) 710 protruding into the grooves 706. Correspondingly, the needle 704 includes multiple notches 708, sized and spaced to accept the ridges 710. The ridges 710 may interlock with the notches 708, producing improved grip, and control. Additionally, the disclosed arrangement prevents relative displacement of the needle 704 within the grooves 706. The needle 704 may further have a distal end 714A and a proximal end 714B. The upper arm (not shown) may also include grooves and ridges similar to the grooves 706 and ridges 710, for increasing the firmness with which the needle 704 is retained in grooves 706. Because the needle 704 is held rigidly in position, the operator can be confident that it will not come loose or slide during preliminary navigation or the suturing itself.

FIG. 7B is a top view of an inner face 716 of a lower arm 718 of the needle driver end-effector 700B, in accordance with yet another embodiment of the present disclosure. Though FIG. 7A shows multiple ridges 710, other embodiments may provide grooves 706 that may include only one ridge 710. Also, the multiple ridges 710 in FIG. 7A are oriented longitudinally, parallel to the long axis of inner face 716. Here, ridge 710 is positioned at the bottom of groove 706, oriented vertically. That position is seen more clearly in FIG. 7C, a cross-sectional view of the needle driver 700B holding the needle 704. Additionally, some embodiments may include groove 706 radiused in a vertical plane, with the radius of groove 706 matching the radius of a needle 704. During the course of grasping a needle 704, radiused groove 706 imposes forces on the needle 704 that urge it into a “right side up” orientation, as shown. Thus, the radiused 706 ensures that a grasped needle is ready for use immediately upon being grasped.

FIG. 8 is a detailed perspective view of a portion of an exemplary face of one arm 802 of a needle driver end-effector 800. This embodiment also includes grooves 804 formed on the surface of the end-effector faces, but here the grooves 804 specifically correspond to and accept a particular needle cross-section. As illustrated, an inner face of the arm 802 i.e. a lower arm 802 of the needle driver end-effector 800 includes “V”-shaped slots or grooves 804 for holding correspondingly configured needles. Thus, in an embodiment, a needle having a diamond shaped cross section would fit perfectly into slots formed by the arm 802 and a corresponding arm (not shown) having matching slots. It will be understood that an end-effector may be designed to accommodate a needle having an asymmetrical profile, in which the lower arm and the upper arm are respectively designed to accept one of the profile shapes.

FIGS. 9A-9B illustrate another exemplary needle driver end-effector 900, in accordance with an embodiment of the present disclosure. The end-effector 900 is similar to the needle driver end-effector 100, as shown in FIGS. 1A-1B, with a structure similar to that of the needle driver end-effector 100. The substantially flat surface 109 of the end-effector 900 includes a knurled zone 902, forming a contacting area to grasp the suture. The knurling can be performed in any suitable geometrical shape for increasing friction, employing as diamond-shaped, triangular, square, and or other configuration, as desired. In an embodiment, the needle driver end-effector 900 may include serration grooves perpendicular to suture for better gripping of the suture. The knurling on the knurled zone 902 may reduce the likelihood of the sure slipping. The knurling may be also used to grip and control straight needle 904, as shown in FIG. 9B. The needle driver end-effector 900 may also have geometric features that can mate with corresponding features on the needle to decrease chances of needle slipping free.

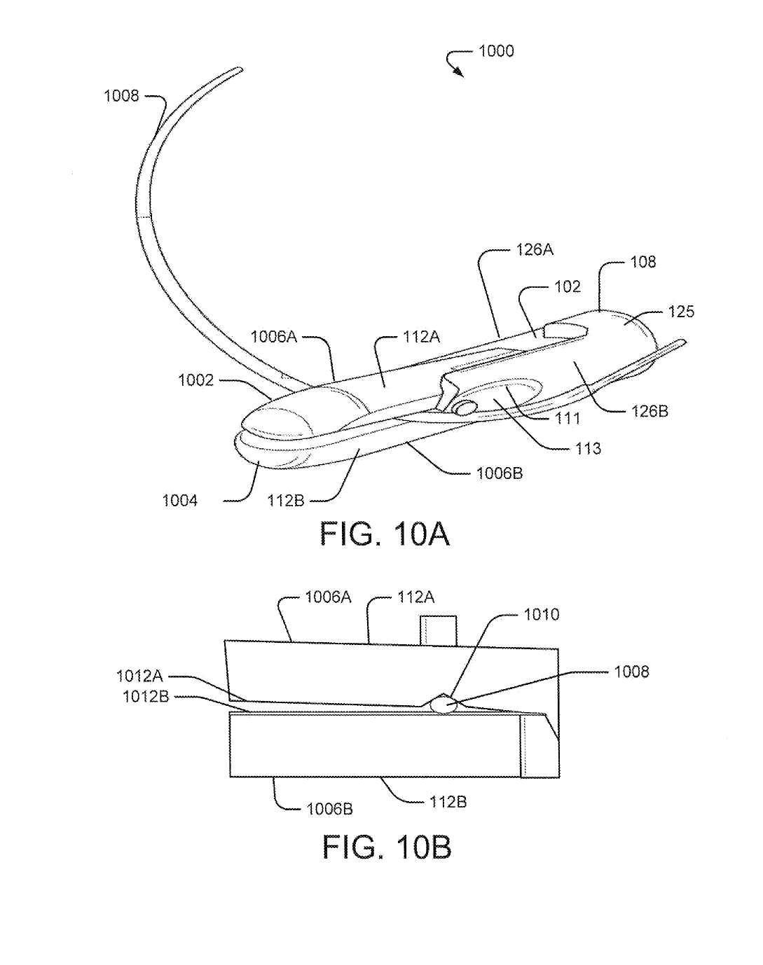

FIG. 10A illustrates another exemplary needle driver end-effector 1000, in accordance with another embodiment of the present disclosure. The end-effector 1000 is similar to the needle driver end-effector 100, as shown in FIGS. 1A-1B, with a structure similar to that of the needle driver end-effector 100. The needle driver end-effector 1000 may have at least two arms—an upper arm 1006A and a lower arm 1006B. The upper arm 1006A of the needle driver end-effector 1000 may have a rounded (or radiused) distal portion 1002. The needle driver end-effector 1000 also have a lower arm 1006B having a scalloped distal portion 1004. In an embodiment, the distal portion 1004 may be underslung, extending slightly distally of the distal portion 1002 of the upper arm 1006A. Each of the arms 1006A-1006B may have an inner face 1012A-1012B, respectively. Further, inner faces 1012A-1012B may have one or more radiused cut(s) 1010 corresponding to the radius of a needle 1008, which would right-side up the needle 1008 as shown in FIG. 10B. FIG. 10B shows a side view showing a radiused cut 1010. The needle driver end-effector 1000 may further include an interlocking keying feature to lock the needle 1008 in position, and prevent rolling or sliding of the needle 1008. The interlocking keying feature may be an interlock mechanism (not shown) that may lock the arms 1006A-1006B closed with a simple locking/unlocking lever or a control member that may be activated by a push-pull wire from a proximal end of the needle driver end-effector 1000.

In some embodiments, the needle driver end-effector 1000 may have an upper arm 1006A having a profile thinner than those of embodiments discussed above. That profile offers a reduced surface area of the upper arm 1006A, which may allow for a higher force holding force. Further, in an embodiment, a high durometer elastomer or plastic on the inner faces 1012A-1012B of the arms 1006A-1006B may aid the gripping of the needle 1008 or a suture. In further embodiments, a portion of the needle may include ridges, curves, or other surface and/or shape irregularities that are configured to be compressed, when grippe in, e.g., a groove. It is contemplated that such irregularities may improve the frictional engagement between the needle and the gripper and improve the retention force of the needle within the gripper.

Other embodiments of the present disclosure will be apparent to those skilled in the art from consideration of the specification and practice of the embodiments disclosed herein. It is intended that the specification and examples be considered as exemplary only, with a true scope and spirit of the invention being indicated by the following claims.

What is claimed is:

1. A needle driver comprising:
an elongated member having a proximal end, a distal end, and a lumen extending therebetween; and
an end-effector disposed at the distal end of the elongated member, the end-effector including at least two arms connected at their proximal ends to form a jaw-like structure, wherein at least one of the at least two arms is configured to rotate relative to the other arm;
wherein each arm has an inner face, the inner face of at least one of the arms including at least one groove configured to receive a portion of a needle, wherein the groove includes a first geometric structure formed in a portion of the groove.

2. The needle driver of claim 1, wherein the at least one groove includes a plurality of grooves.

3. The needle driver of claim 1, wherein the inner face of at least one of the arms includes a substantially flat surface disposed adjacent the at least one groove.

4. The needle driver of claim 2, wherein the needle is an arcuate needle and each of the plurality of grooves is curved to correspond to the arcuate needle.

5. The needle driver of claim 1, wherein the first geometric structure is a projection.

6. The needle driver of claim 1, wherein the groove includes a plurality of first geometric structures.

7. The needle driver of claim 1, wherein the first geometric structure is configured to mate with a corresponding second geometric structure formed in a portion of the needle.

8. The needle driver of claim 7, wherein the second geometric structure is a cavity configured to receive the first geometric structure.

9. The needle driver of claim 7, wherein the needle includes a plurality of second geometric structures.

10. The needle driver of claim 1, wherein both arms of the end-effector are configured to rotate relative to one another.

11. A needle driver comprising:
an end-effector disposed at the distal end of the elongated member, the end-effector including at least two arms pivotally connected at their proximal ends;
wherein each arm has an inner face, the inner face of at least one of the arms having a plurality of grooves configured to receive a portion of a needle and a substantially flat surface disposed adjacent the plurality of grooves, and wherein each of the plurality of grooves includes a projection configured to mate with a corresponding recess on the needle.

12. The needle driver of claim 11, wherein the needle is an arcuate needle and each of the plurality of grooves is curved to correspond to the arcuate needle.

13. The needle driver of claim 11, wherein the projection is a plurality of projections.

14. The needle driver of claim 11, wherein both arms of the end-effector are configured to rotate relative to one another.

15. The needle driver of claim 11, wherein the end-effector is secured to a distal end of an endoscopic tool.

16. The needle driver of claim 11, wherein the needle is configured to be securely attached to a length of suture.

17. The needle driver of claim 14, wherein the substantially flat surface includes surface features to increase friction during gripping.

18. A needle driver comprising:
an elongated member having a proximal end, a distal end, and a lumen extending therebetween; and
an end-effector disposed at the distal end of the elongated member, the end-effector including two arms connected at their proximal ends to form a jaw-like structure, wherein each of the two arms is configured to rotate relative to the other arm;
wherein each arm has an inner face, the inner face of both arms including a plurality of groove configured to receive a portion of a needle, wherein each of the plurality of grooves includes a first geometric structure configured to mate with a corresponding second geometric structure on the needle.

19. The needle driver of claim 18, wherein each of the plurality of grooves includes a plurality of geometric structures.

20. The needle driver of claim 18, wherein each of the inner faces includes a substantially flat surface disposed adjacent to the plurality of grooves.
